(19) United States
(12) Patent Application Publication
(10) Pub. No.: US 2012/0202281 A1
(43) Pub. Date: Aug. 9, 2012

(54) LIGHT ENERGY SUPPLY FOR PHOTOBIOREACTOR SYSTEM

(75) Inventors: Jaime A. Gonzalez, Oakville (CA); Max Kolesnik, Schomberg (CA); Steven C. Martin, Toronto (CA)

(73) Assignee: POND BIOFUELS INC., Scarborough (CA)

(21) Appl. No.: 13/022,508

(22) Filed: Feb. 7, 2011

(51) Int. Cl. C12M 1/00

(52) U.S. Cl. 435/292.1

(57) ABSTRACT

Photobioreactor systems including a supply material processing sub-system, a reactor sub-system, a product material processing sub-system, and a solar energy supply sub-system are provided. The reactor sub-system includes a photobioreactor configured for containing a reaction mixture that is operative for effecting photosynthesis upon exposure to photosynthetically active light radiation. The supply material processing sub-system is configured for supplying the reactor with supply material. The product material processing sub-system is configured for receiving reaction zone product discharged from the reactor and effecting separation of a liquid component from the received reaction zone product. The solar energy supply sub-system includes at least one solar collector mounted to the photobioreactor and includes a solar collector surface configured for receiving incident solar radiation and configured for transmitting energy derived from received incident solar radiation and supplying energy to at least one of the other sub-systems.

### TECHNICAL FIELD

The present disclosure relates to photobioreactors that utilize incident solar radiation.

### BACKGROUND

The cultivation of phototrophic organisms has been widely practised for purposes of producing a fuel source. Exhaust gases from industrial processes have also been used to promote the growth of phototrophic organisms by supplying carbon dioxide for consumption by phototrophic organisms during photosynthesis. By providing exhaust gases for such purpose, environmental impact is reduced and, in parallel a potentially useful fuel source is produced. Challenges remain, however, to render this approach more economically attractive for incorporation within existing facilities.

### SUMMARY

In one aspect, there is provided a photobioreactor system including a supply material processing sub-system, a reactor sub-system, a product material processing sub-system, and a solar energy supply sub-system. The reactor sub-system includes a photobioreactor configured for containing a reaction mixture that is operative for effecting photosynthesis upon exposure to photosynthetically active light radiation, wherein the reaction mixture includes photosynthesis reaction reagents. The supply material processing sub-system is configured for supplying the reactor with supply material, wherein the supply material includes at least one of the photosynthesis reaction reagents. The product material processing sub-system is configured for receiving reaction zone product discharged from the reactor and effecting separation of a liquid component from the received reaction zone product. The solar energy supply sub-system includes at least one solar collector, wherein each one of the at least one solar collector is mounted to the photobioreactor and includes a solar collector surface configured for receiving incident solar radiation such that at least one solar collector surface is provided to define a total photobioreactor-connected solar collector surface area, wherein each one of the at least one solar collector is operatively coupled to an energy supply component that is configured for transmitting energy derived from the received incident solar radiation and supplying the energy to at least one of the other sub-systems. The total photobioreactor-connected solar collector surface area is at least 75 square metres.

In another aspect, there is provided a photobioreactor system including a supply material processing sub-system, a reactor sub-system, a product material processing sub-system, and a solar energy supply sub-system. The reactor sub-system includes a photobioreactor configured for containing a reaction mixture that is operative for effecting photosynthesis upon exposure to photosynthetically active light radiation, wherein the reaction mixture includes photosynthesis reaction reagents. The supply material processing sub-system is configured for supplying the reactor with supply material, wherein the supply material includes at least one of the photosynthesis reaction reagents. The product material processing sub-system is configured for receiving reaction zone product discharged from the reactor and effecting separation of a liquid component from the received reaction zone product. The solar energy supply sub-system includes at least one solar collector, wherein each one of the at least one solar collector is mounted to an operative mounting surface of the photobioreactor and includes a solar collector surface configured for receiving incident solar radiation, wherein each one of the at least one solar collector is operatively coupled to an energy supply component that is configured for transmitting energy derived from the received incident solar radiation and supplying the energy to at least one of the other sub-systems. The operative mounting surface is oriented within 45 degrees of the vertical.

In another aspect, there is provided a photobioreactor comprising a container and a plurality of operative light transmission components. The container is configured for containing a reaction mixture in a reaction zone, wherein the reaction mixture is operative for effecting photosynthesis upon exposure to photosynthetically active light radiation, and includes photosynthesis reaction reagents, and wherein the reaction zone includes a volume of at least 3000 litres. The plurality of operative light transmission components configured for supplying light energy to the reaction zone of the photobioreactor to thereby effect exposure of the reaction mixture within at least 80% of the reaction zone to photosynthetically active light radiation, wherein each one of the operative light transmission components is mounted to and extends into the reaction zone from an operative portion of an internal surface of the photobioreactor for an operative distance, wherein the operative distance is less than five (5) metres.

In another aspect, there is provided a photobioreactor system including a supply material processing sub-system, a reactor sub-system, a product material processing sub-system, and a solar energy supply sub-system. The reactor sub-system includes a photobioreactor configured for containing a reaction mixture that is operative for effecting photosynthesis upon exposure to photosynthetically active light radiation, wherein the reaction mixture includes photosynthesis reaction reagents. The supply material processing sub-system is configured for supplying the reactor with supply material, wherein the supply material includes at least one of the photosynthesis reaction reagents. The product material processing sub-system is configured for receiving reaction zone product discharged from the reactor and effecting separation of a liquid component from the received reaction zone product. The solar energy supply sub-system includes at least one solar collector, wherein each one of the at least one solar collector includes a solar collector surface configured for receiving incident solar radiation, and also including a plurality of vertically spaced energy supply components, wherein each one of the vertically spaced energy supply components is configured for transmitting energy derived from the received incident solar radiation and supplying the energy to at least one of the other sub-systems, wherein each one of the at least one solar collector is operatively coupled to at least one of the vertically spaced energy supply components. Each one of the vertically spaced energy supply components extends into the reaction zone from an operative portion of an internal surface of the photobioreactor, wherein the internal surface of the photobioreactor defines a space including the reaction zone. Each one of the vertically spaced energy supply components is disposed at a different vertical position relative to the other ones of the vertically spaced energy supply components.

### BRIEF DESCRIPTION OF THE DRAWINGS

The process of the preferred embodiments of the invention will now be described with the following accompanying drawings:

FIG. 1 is a block diagram of an embodiment of the photobioreactor system;

FIG. 2 is a process flow diagram of an embodiment of a process that is operational in an embodiment of the photobioreactor system;

FIG. 3 is a process flow diagram of another embodiment of a process that is operational in an embodiment of the photobioreactor system;

FIG. 4 is a schematic illustration of an embodiment of the photobioreactor system including a plurality of solar collectors;

FIG. 5 is a schematic illustration of an embodiment of the photobioreactor system including a solar collector that includes a filter/mirror assembly 1006 that filters incident solar radiation received by the solar collector to provide a light source-purpose received incident solar radiation fraction and a power generation-purpose received incident solar radiation fraction;

FIG. 6 is a schematic illustration of another embodiment of the photobioreactor system including a solar collector that includes a filter/mirror assembly 1006 that filters incident solar radiation received by the solar collector to provide a light source-purpose received incident solar radiation fraction and a power generation-purpose received incident solar radiation fraction;

FIG. 7 is a schematic illustration of another embodiment of the photobioreactor system including a plurality of solar collectors; and

FIG. 8 is a schematic illustration of a portion of a fluid passage of an embodiment of the process.

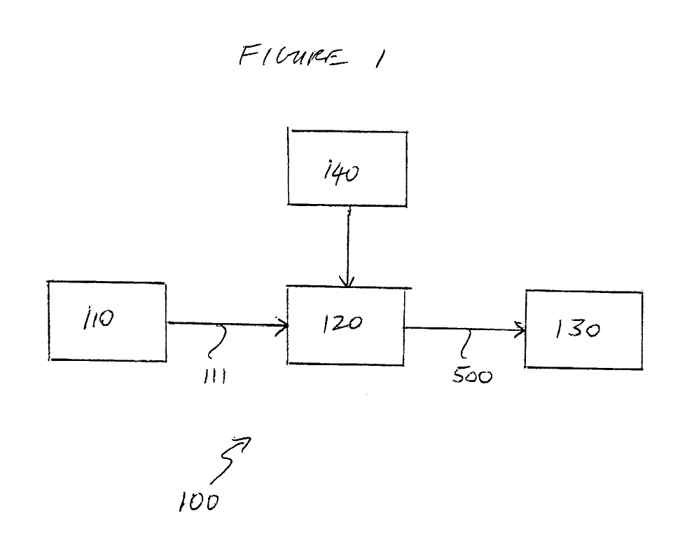

### DETAILED DESCRIPTION

Reference throughout the specification to “some embodiments” means that a particular feature, structure, or characteristic described in connection with some embodiments are not necessarily referring to the same embodiments. Furthermore, the particular features, structure, or characteristics may be combined in any suitable manner with one another.

“Phototrophic organism” is an organism capable of phototrophic growth in the aqueous medium upon receiving light energy, such as plant cells and micro-organisms. The phototrophic organism is unicellular or multicellular. In some embodiments, for example, the phototrophic organism is an organism which has been modified artificially or by gene manipulation. In some embodiments, for example, the phototrophic organism is an alga. In some embodiments, for example, the algae are microalgae.

“Phototrophic biomass” is at least one phototrophic organism. In some embodiments, for example, the phototrophic biomass includes more than one species of phototrophic organisms.

“Reaction zone 10” defines a space within which the growing of the phototrophic biomass is effected. In some embodiments, for example, the reaction zone 10 is provided in a photobioreactor 12. In some embodiments, for example, pressure within the reaction zone is atmospheric pressure.

“Photobioreactor 12” is any structure, arrangement, land formation or area that provides a suitable environment for the growth of phototrophic biomass. Examples of specific structures which can be used is a photobioreactor 12 by providing space for growth of phototrophic biomass using light energy include, without limitation, tanks, ponds, troughs, ditches, pools, pipes, tubes, canals, and channels. Such photobioreactors may be either open, closed, partially closed, covered, or partially covered. In some embodiments, for example, the photobioreactor 12 is a pond, and the pond is open, in which case the pond is susceptible to uncontrolled receiving of materials and light energy from the immediate environments. In other embodiments, for example, the photobioreactor 12 is a covered pond or a partially covered pond, in which case the receiving of materials from the immediate environment is at least partially interfered with. The photobioreactor 12 includes the reaction zone 10 which includes the reaction mixture. In some embodiments, the photobioreactor 12 is configured to receive a supply of phototrophic reagents (and, in some of these embodiments, optionally, supplemental nutrients), and is also configured to effect discharge of phototrophic biomass which is grown within the reaction zone 10. In this respect, in some embodiments, the photobioreactor 12 includes one or more inlets for receiving the supply of phototrophic reagents and supplemental nutrients, and also includes one or more outlets for effecting the recovery or harvesting of biomass which is grown within the reaction zone 10. In some embodiments, for example, one or more of the inlets are configured to be temporarily sealed for periodic or intermittent time intervals. In some embodiments, for example, one or more of the outlets are configured to be temporarily sealed or substantially sealed for periodic or intermittent time intervals. The photobioreactor 12 is configured to contain the reaction mixture which is operative for effecting photosynthesis upon exposure to photosynthetically active light radiation. The photobioreactor 12 is also configured so as to establish photosynthetically active light radiation (for example, a light of a wavelength between about 400-700 nm, which can be emitted by the sun or another light source) within the photobioreactor 12 for exposing the phototrophic biomass. The exposing of the reaction mixture to the photosynthetically active light radiation effects photosynthesis and growth of the phototrophic biomass. In some embodiments, for example, the established light radiation is provided by an artificial light source 14 disposed within the photobioreactor 12. For example, suitable artificial lights sources include submersible fiber optics or light guides, light-emitting diodes (“LEDs”), LED strips and fluorescent lights. Any LED strips known in the art can be adapted for use in the photobioreactor 12. In the case of the submersible LEDs, in some embodiments, for example, energy sources include alternative energy sources, such as wind, photovoltaic cells, fuel cells, etc. to supply electricity to the LEDs. Fluorescent lights, external or internal to the photobioreactor 12, can be used as a back-up system. In some embodiments, for example, the established light is derived from a natural light source 16 which has been transmitted from externally of the photobioreactor 12 and through a transmission component. In some embodiments, for example, the transmission component is a portion of a containment structure of the photobioreactor 12 which is at least partially transparent to the photosynthetically active light radiation, and which is configured to provide for transmission of such light to the reaction zone 10 for receiving by the phototrophic biomass. In some embodiments, for example, natural light is received by a solar collector, filtered with selective wavelength filters, and then transmitted to the reaction zone 10 with fiber optic material or with a light guide. In some embodiments, for example, both natural and artificial lights sources are provided for effecting establishment of the photosynthetically active light radiation within the photobioreactor 12.

“Aqueous medium” is an environment that includes water. In some embodiments, for example, the aqueous medium also includes sufficient nutrients to facilitate viability and growth of the phototrophic biomass. In some embodiments, for example, supplemental nutrients may be included such as one of, or both of, NO_{X} and SO_{X}. Suitable aqueous media are discussed in detail in: Rogers, L. J. and Gallon J. R. “Biochemistry of the Algae and Cyanobacteria,” Clarendon Press Oxford, 1988; Burlew, John S. “Algal Culture: From Laboratory to Pilot Plant.” Carnegie Institution of Washington Publication 600. Washington, D.C., 1961 (hereinafter “Burlew 1961”); and Round, F. E. The Biology of the Algae. St Martin's Press, New York, 1965; each of which is incorporated herein by reference). A suitable supplemental nutrient composition, known as “Bold's Basal Medium”, is described in Bold, H. C. 1949, The morphology of Chlamydomonas chlamydogama sp. nov. Bull. Torrey Bot. Club. 76:101-8 (see also Bischoff, H. W. and Bold, H. C. 1963. Phycological Studies IV. Some soil algae from Enchanted Rock and related algal species, Univ. Texas Publ. 6318:1-95, and Stein, J. (ED.) Handbook of Phycological Methods, Culture methods and growth measurements, Cambridge University Press, pp. 7-24).

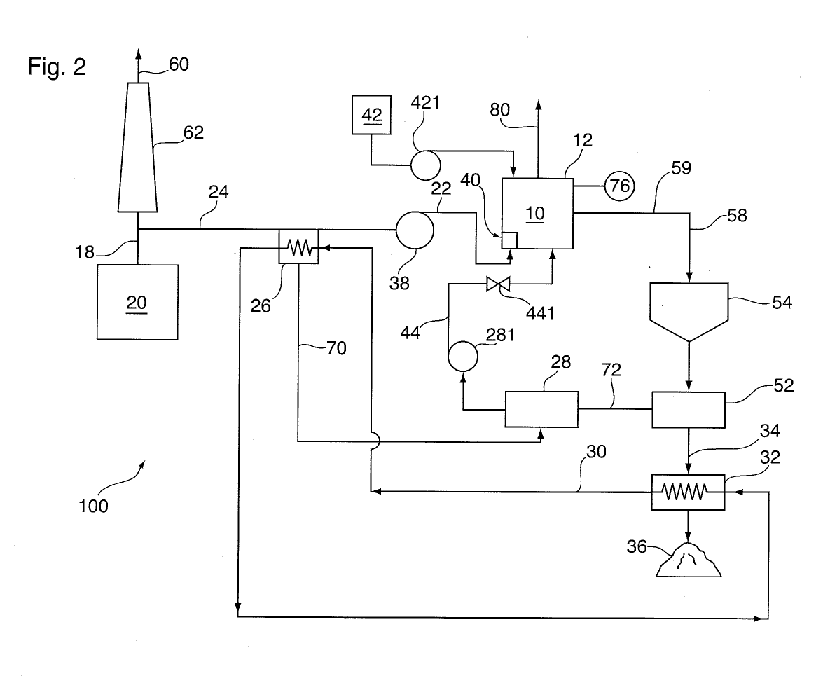

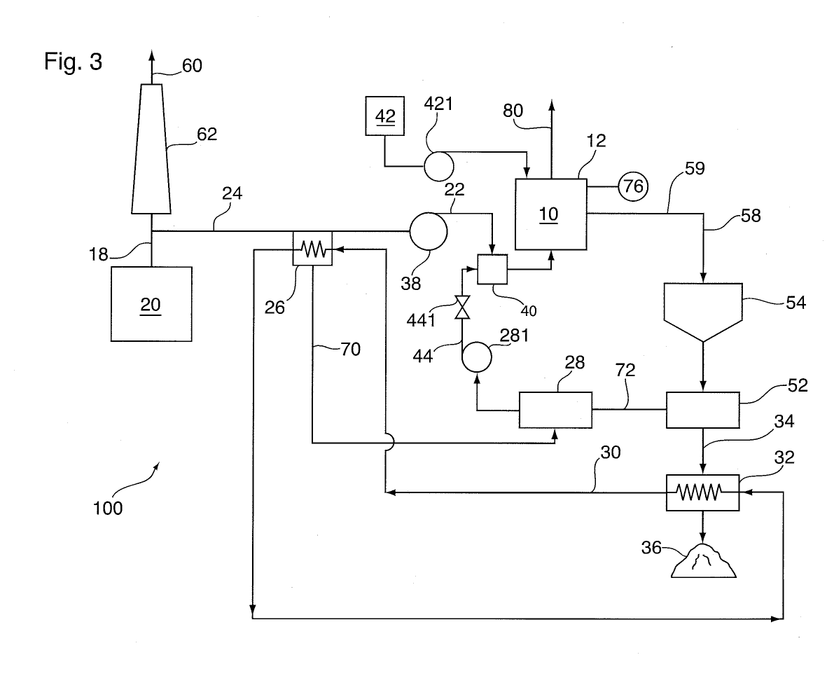

There is provided a photobioreactor system 100. Exemplary processes of growing a phototrophic biomass that are operational in embodiments of the photobioreactor system 100 are illustrated in FIGS. 2 and 3.

The reaction zone 10 includes a reaction mixture that is operative for effecting photosynthesis upon exposure to photosynthetically active light radiation. The reaction mixture includes photosynthesis reaction reagents. The photosynthesis reaction reagents include phototrophic biomass material, carbon dioxide, and water. In some embodiments, the reaction zone includes phototrophic biomass and carbon dioxide disposed in an aqueous medium. Within the reaction zone, the phototrophic biomass is disposed in mass transfer communication with both of carbon dioxide and water.

The photobioreactor system 100 includes a material supply sub-system 110, a reactor sub-system 120, a product material processing sub-system 130, and a solar energy supply sub-system 140.

The material supply sub-system 110 is configured for supplying material 111 to a reaction zone 10 of the reactor sub-system 120. Material that is supplied by the material supply sub-system includes one or more of the photosynthesis reaction reagents. In some embodiments, the supplied photosynthesis reaction reagents include water and carbon dioxide. In some embodiments, each of water and carbon dioxide is separately introduced into the reactor sub-system 120. In other embodiments, water and carbon dioxide are introduced in the form of an aqueous mixture. The photosynthesis reaction reagents being supplied define the respective process material component of the supply material processing-subsystem. In some embodiments, for example, the supplied material also includes supplemental nutrient supply 42.

In some embodiments, for example, the carbon dioxide supplied to the reaction zone 10 is a gaseous exhaust material 18. In this respect, in some embodiments, the carbon dioxide is supplied by a gaseous exhaust material producing process 20, and the supplying is, therefore, effected by producing the gaseous exhaust material 18 with a gaseous exhaust material producing process 20. The gaseous exhaust material 18 includes carbon dioxide. The gaseous exhaust material producing process 20 includes any process which effects production of the gaseous exhaust material. In some embodiments, for example, the gaseous exhaust material producing process 20 is a combustion process being effected in a combustion facility. In some of these embodiments, for example, the combustion process effects combustion of a fossil fuel, such as coal, oil, or natural gas. For example, the combustion facility is any one of a fossil fuel-fired power plant, an industrial incineration facility, an industrial furnace, an industrial heater, or an internal combustion engine. In some embodiments, for example, the combustion facility is a cement kiln.

Reaction zone feed material 22 is supplied to the reaction zone 10 such that any carbon dioxide of the reaction zone feed material 22 is received within the reaction zone 10. During at least some periods of operation of the process, at least a fraction of the reaction zone feed material 22 is supplied by the gaseous exhaust material 18 which is discharged from the gaseous exhaust material producing process 20. Any gaseous exhaust material 18 that is supplied to the reaction zone feed material 22 is supplied as a gaseous exhaust material reaction zone supply 24. It is understood that not the entirety of the gaseous exhaust material 18 is necessarily supplied to the gaseous exhaust material reaction zone supply 24, or at least not for the entire time period during which the process is operational. The gaseous exhaust material reaction zone supply 24 includes carbon dioxide. In some embodiments, for example, the gaseous exhaust material 18 includes a carbon dioxide concentration of at least 2 volume % based on the total volume of the gaseous exhaust material 18. In this respect, in some embodiments, for example, the gaseous exhaust material reaction zone supply 24 includes a carbon dioxide concentration of at least 2 volume % based on the total volume of the gaseous exhaust material reaction zone supply 24. In some embodiments, for example, the gaseous exhaust material 18 includes a carbon dioxide concentration of at least 4 volume % based on the total volume of the gaseous exhaust material 18. In this respect, in some embodiments, for example, the gaseous exhaust material reaction zone supply 24 includes a carbon dioxide concentration of at least 4 volume % based on the total volume of the gaseous exhaust material reaction zone supply 24. In some embodiments, for example, the gaseous exhaust material reaction zone supply 24 also includes one of, or both of, NO_{X} and SO_{X}.

In some of these embodiments, for example, the gaseous exhaust material reaction zone supply 24 is at least a fraction of the gaseous exhaust material 18 being produced by the gaseous exhaust material producing process 20. In some cases, the gaseous exhaust material reaction zone supply 24 is the gaseous exhaust material 18 being produced by the gaseous exhaust material producing process 20.

In some embodiments, for example, at least a fraction of the gaseous exhaust material 18 being produced by the gaseous exhaust material producing process 20 is supplied to another unit operation 62 as a bypass gaseous exhaust material 60. The bypass gaseous exhaust material 60 includes carbon dioxide. The another unit operation converts the bypass gaseous exhaust material 60 such that its environmental impact is reduced. In some embodiments, for example, the another unit operation 62 is a smokestack.

In some embodiments, for example, the reaction zone feed material 22 is cooled prior to supply to the reaction zone 10 so that the temperature of the reaction zone feed material 22 aligns with a suitable temperature at which the phototrophic biomass can grow. In some embodiments, for example, the gaseous exhaust material reaction zone supply 24 being supplied to the reaction zone material 22 is disposed at a temperature of between 110 degrees Celsius and 150 degrees Celsius. In some embodiments, for example, the temperature of the gaseous exhaust material reaction zone supply 24 is about 132 degrees Celsius. In some embodiments, the temperature at which the gaseous exhaust material reaction zone supply 24 is disposed is much higher than this, and, in some embodiments, such as the gaseous exhaust material reaction zone supply 24 from a steel mill, the temperature is over 500 degrees Celsius. In some embodiments, for example, the reaction zone feed material 22, which has been supplied with the gaseous exhaust material reaction zone supply 24, is cooled to between 20 degrees Celsius and 50 degrees Celsius (for example, about 30 degrees Celsius). Supplying the reaction zone feed material 22 at higher temperatures could hinder growth, or even kill the phototrophic biomass in the reaction zone 10. In some of these embodiments, in cooling the reaction zone feed material 22, at least a fraction of any water vapour in the reaction zone feed material 22 is condensed in a heat exchanger 26 (such as a condenser) and separated from the reaction zone feed material 22 as an aqueous material 70. In some embodiments, the resulting aqueous material 70 is diverted to a container 28 (described below) where it provides supplemental aqueous material supply 44 for supply to the reaction zone 10. In some embodiments, the condensing effects heat transfer from the reaction zone feed material 22 to a heat transfer medium 30, thereby raising the temperature of the heat transfer medium 30 to produce a heated heat transfer medium 30, and the heated heat transfer medium 30 is then supplied (for example, flowed) to a dryer 32 (discussed below), and heat transfer is effected from the heated heat transfer medium 30 to an intermediate concentrated reaction zone product 34 to effect drying of the intermediate concentrated reaction zone product 34 and thereby effect production of the final reaction zone product 36. In some embodiments, for example, after being discharged from the dryer 32, the heat transfer medium 30 is recirculated to the heat exchanger 26. Examples of a suitable heat transfer medium 30 include thermal oil and glycol solution.

With respect to the reaction zone feed material 22, the reaction zone feed material 22 is a fluid. In some embodiments, for example, the reaction zone feed material 22 is a gaseous material. In some embodiments, for example, the reaction zone feed material 22 includes gaseous material disposed in liquid material. In some embodiments, for example, the liquid material is an aqueous material. In some of these embodiments, for example, at least a fraction of the gaseous material is dissolved in the liquid material. In some of these embodiments, for example, at least a fraction of the gaseous material is disposed as a gas dispersion in the liquid material. In some of these embodiments, for example, and during at least some periods of operation of the process, the gaseous material of the reaction zone feed material 22 includes carbon dioxide supplied by the gaseous exhaust material reaction zone supply 24. In some of these embodiments, for example, the reaction zone feed material 22 is supplied to the reaction zone 10 as a flow.

In some embodiments, for example, the reaction zone feed material 22 is supplied to the reaction zone 10 as one or more reaction zone feed material flows. For example, each of the one or more reaction zone feed material flows is flowed through a respective reaction zone feed material fluid passage. In some of those embodiments where there are more than one reaction zone feed material flow, the material composition varies between the reaction zone feed material flows. In some embodiments, for example, a flow of reaction zone feed material 22 includes a flow of the gaseous exhaust material reaction zone feed material supply 24. In some embodiments, for example, a flow of reaction zone feed material 22 is a flow of the gaseous exhaust material reaction zone feed material supply 24.

In some embodiments, for example, the supply of the reaction zone feed material 22 to the reaction zone 10 effects agitation of at least a fraction of the phototrophic biomass disposed in the reaction zone 10. In this respect, in some embodiments, for example, the reaction zone feed material 22 is introduced to a lower portion of the reaction zone 10. In some embodiments, for example, the reaction zone feed material 22 is introduced from below the reaction zone 10 so as to effect mixing of the contents of the reaction zone 10. In some of these embodiments, for example, the effected mixing (or agitation) is such that any difference in phototrophic biomass concentration between two points in the reaction zone 10 is less than 20%. In some embodiments, for example, any difference in phototrophic biomass concentration between two points in the reaction zone 10 is less than 10%. In some of these embodiments, for example, the effected mixing is such that a homogeneous suspension is provided in the reaction zone 10. In those embodiments with a photobioreactor 12, for some of these embodiments, for example, the supply of the reaction zone feed material 22 is co-operatively configured with the photobioreactor 12 so as to effect the desired agitation of the at least a fraction of the phototrophic biomass disposed in the reaction zone 10.

With further respect to those embodiments where the supply of the reaction zone feed material 22 to the reaction zone 10 effects agitation of at least a fraction of the phototrophic biomass disposed in the reaction zone 10, in some of these embodiments, for example, the reaction zone feed material 22 flows through a gas injection mechanism, such as a sparger 40, before being introduced to the reaction zone 10. In some of these embodiments, for example, the sparger 40 provides reaction zone feed material 22 as a gas-liquid mixture to the reaction zone 10 in fine bubbles in order to maximize the interface contact area between the phototrophic biomass and the carbon dioxide (and, in some embodiments, for example, one of, or both of, SO_{X} and NO_{X}) of the reaction zone feed material 22. This assists the phototrophic biomass in efficiently absorbing the carbon dioxide (and, in some embodiments, other gaseous components) required for photosynthesis, thereby promoting the optimization of the growth rate of the phototrophic biomass. As well, in some embodiments, for example, the sparger 40 provides reaction zone feed material 22 in larger bubbles that agitate the phototrophic biomass in the reaction zone 10 to promote mixing of the components of the reaction zone 10. An example of a suitable sparger 40 is EDI FlexAir™ T-Series Tube Diffuser Model 91 X 1003 supplied by Environmental Dynamics Inc. of Columbia, Mo. In some embodiments, for example, this sparger 40 is disposed in a photobioreactor 12 (see FIG. 2) having a reaction zone 10 volume of 6000 litres and with an algae concentration of between 0.8 grams per litre and 1.5 grams per litre, and the reaction zone feed material 22 is a gaseous fluid flow supplied at a flow rate of between 10 cubic feet per minute and 20 cubic feet per minute, and at a pressure of about 68 inches of water.

With respect to the sparger 40, in some embodiments, for example, the sparger 40 is designed to consider the fluid head of the reaction zone 10, so that the supplying of the reaction zone feed material 22 to the reaction zone 10 is effected in such a way as to promote the optimization of carbon dioxide absorption by the phototrophic biomass. In this respect, bubble sizes are regulated so that they are fine enough to promote optimal carbon dioxide absorption by the phototrophic biomass from the reaction zone feed material. Concomitantly, the bubble sizes are large enough so that at least a fraction of the bubbles rise through the entire height of the reaction zone 10, while mitigating against the reaction zone feed material 22 “bubbling through” the reaction zone 10 and being released without being absorbed by the phototrophic biomass. To promote the realization of an optimal bubble size, in some embodiments, the pressure of the reaction zone feed material 22 is controlled using a pressure regulator upstream of the sparger 40.

With respect to those embodiments where the reaction zone 10 is disposed in a photobioreactor 12, in some of these embodiments, for example, the sparger 40 is disposed externally of the photobioreactor 12 (see FIG. 3). In other embodiments, for example, the sparger 40 is disposed within the photobioreactor 12. In some of these embodiments, for example, the sparger 40 extends from a lower portion of the photobioreactor 12 (and within the photobioreactor 12).

In some embodiments, for example, the reaction zone feed material 22 is supplied at a pressure which effects flow of the reaction zone feed material 22 through at least a seventy (70) inch vertical extent of the reaction zone. In some embodiments, for example, the vertical extent is at least 10 feet. In some embodiments, for example, the vertical extent is at least 20 feet. In some embodiments, for example, the vertical extent is at least 30 feet. In some of these embodiments, for example, the supplying of the reaction zone feed material 22 is effected while the gaseous exhaust material 18 is being produced by the gaseous exhaust material producing process 20 and while at least a fraction of the gaseous exhaust material 18 is being supplied to the reaction zone feed material 22 (as the gaseous exhaust material reaction zone supply 24). In some of these embodiments, for example, the pressure of the material of a flow of the gaseous exhaust material reaction zone supply 24 (whether by itself or as a portion of the flow of the reaction zone feed material 22) is increased before being supplied to the reaction zone 10. In some embodiments, for example, the pressure increase is at least partially effected by a prime mover 38. For those embodiments where the pressure increase is at least partially effected by the prime mover 38. An example of a suitable prime mover 38, for embodiments where the gaseous exhaust material reaction zone supply 24 is a portion of a flow of the reaction zone feed material 22, and the reaction zone feed material includes liquid material, is a pump. Examples of a suitable prime mover 38, for embodiments where the pressure increase is effected to a gaseous flow, include a blower, a compressor, and an air pump. In other embodiments, for example, the pressure increase is effected by a jet pump or eductor. With respect to such embodiments, where the pressure increase is effected by a jet pump or eductor, in some of these embodiments, for example, the gaseous exhaust material reaction zone supply 24 is supplied to the jet pump or eductor and pressure energy is transferred to the gaseous exhaust material reaction zone from another flowing fluid (the “motive fluid flow”) using the venturi effect to effect a pressure increase in the gaseous exhaust material reaction zone supply 24 component of the reaction zone feed material 22. In this respect, in some embodiments, for example, and referring to FIG. 3, a motive fluid flow 700 is provided, wherein material of the motive fluid flow 700 includes a motive fluid pressure P_{M1}, wherein P_{M1} is greater than the pressure (P_{E}) of the gaseous exhaust material reaction zone supply 24. Pressure of the motive fluid flow 700 is reduced from P_{M1} to P_{M2} by flowing the motive fluid flow 700 from an upstream fluid passage portion 702 to an intermediate downstream fluid passage portion 704. The first intermediate downstream fluid passage portion 704 is characterized by a smaller cross-sectional area relative to the upstream fluid passage portion 702. Further, P_{M2} is less than P_{E}. When the pressure of the motive fluid flow 700 has becomes reduced to P_{M2}, fluid communication between the motive fluid flow 700 and the gaseous exhaust material reaction zone supply 24 is effected such that the material of the gaseous exhaust material reaction zone supply 24 is induced to mix with the motive fluid flow 700 in the intermediate downstream fluid passage portion 704, in response to the pressure differential between the supply 24 and the motive fluid flow 700, to produce a gaseous exhaust material reaction zone supply-derived flow 24A. Pressure of the gaseous exhaust material reaction zone supply-derived flow 24A, which includes the gaseous exhaust material reaction zone supply is increased to P_{M3}, wherein P_{M3} is greater than P_{E}. The pressure increase is effected by flowing the gaseous exhaust material reaction zone supply-derived flow 24A from the intermediate downstream fluid passage portion 704 to a “kinetic energy to static pressure energy conversion” downstream fluid passage portion 706. The cross-sectional area of the “kinetic energy to static pressure energy conversion” downstream fluid passage portion 706 is greater than the cross-sectional area of the intermediate downstream fluid passage portion 704. The gaseous exhaust material reaction zone supply-derived flow 24A, including the gaseous exhaust material reaction zone supply 24, is disposed at a pressure that is greater than P_{E} and that is sufficient to effect flow of material of the flow 24A, as at least a portion of the flow of the reaction zone feed material 22, through at least a seventy (70) inch vertical extent of the reaction zone 10. In some embodiments, for example, a converging nozzle portion of a fluid passage defines the first intermediate downstream fluid passage portion 704 and a diverging nozzle portion of the fluid passage defines the “kinetic energy to static pressure energy conversion” downstream fluid passage portion 706. In some embodiments, for example, the combination of the first intermediate downstream fluid passage portion 704 and the “kinetic energy to static pressure energy conversion” downstream fluid passage portion 706 is defined by a venture nozzle. In some embodiments, for example, the combination of the first intermediate downstream fluid passage portion 704 and the “kinetic energy to static pressure energy conversion” downstream fluid passage portion 706 is disposed within an eductor or jet pump. In some of these embodiments, for example, the motive fluid flow includes liquid aqueous material and, in this respect, the flow 24A includes a combination of liquid and gaseous material. In this respect, in some embodiments, for example, the gaseous exhaust material reaction zone supply-derived flow 24A includes a dispersion of a gaseous material within a liquid material, wherein the dispersion of a gaseous material includes carbon dioxide of the gaseous exhaust material reaction zone supply 24. Alternatively, in some of these embodiments, for example, the motive fluid flow is another gaseous flow, such as an air flow, and the flow 24A is a gaseous flow. The material of the flow 24A is supplied to the reaction zone 10, as at least a portion of a flow of the reaction zone feed material 22, at a pressure greater than P_{E} and sufficient to effect flow of the material of the flow 24A through at least a seventy (70) inch vertical extent of the reaction zone 10. This pressure increase is designed to overcome the fluid head within the reaction zone 10.

In some embodiments, for example, a supplemental nutrient supply 42 is supplied to the reaction zone 10. In some embodiments, for example, the supplemental nutrient supply 42 is effected by a pump, such as a dosing pump. In other embodiments, for example, the supplemental nutrient supply 42 is supplied manually to the reaction zone 10. Nutrients within the reaction zone 10 are processed or consumed by the phototrophic biomass, and it is desirable, in some circumstances, to replenish the processed or consumed nutrients. A suitable nutrient composition is “Bold's Basal Medium”, and this is described in Bold, H. C. 1949, The morphology of Chlamydomonas chlamydogama sp. nov. Bull. Torrey Bot. Club. 76:101-8 (see also Bischoff, H. W. and Bold, H. C. 1963. Phycological Studies IV. Some soil algae from Enchanted Rock and related algal species, Univ. Texas Publ. 6318:1-95, and Stein, J. (ED.) Handbook of Phycological Methods, Culture methods and growth measurements, Cambridge University Press, pp. 7-24). The supplemental nutrient supply 42 is supplied for supplementing the nutrients provided within the reaction zone, such as “Bold's Basal Medium”, or one ore more dissolved components thereof. In this respect, in some embodiments, for example, the supplemental nutrient supply 42 includes “Bold's Basal Medium”. In some embodiments for example, the supplemental nutrient supply 42 includes one or more dissolved components of “Bold's Basal Medium”, such as NaNO₃, CaCl₂, MgSO₄, KH₂PO₄, NaCl, or other ones of its constituent dissolved components.

In some of these embodiments, the rate of supply of the supplemental nutrient supply 42 to the reaction zone 10 is controlled to align with a desired rate of growth of the phototrophic biomass in the reaction zone 10. In some embodiments, for example, regulation of nutrient addition is monitored by measuring any combination of pH, NO₃ concentration, and conductivity in the reaction zone 10.

In some embodiments, for example, a supply of the supplemental aqueous material supply 44 is effected to the reaction zone 10 so as to replenish water within the reaction zone 10 of the photobioreactor 12. In some embodiments, for example, the supplemental aqueous material is water. In some embodiments, for example, the supplemental aqueous material supply 44 includes at least one of: (a) aqueous material 70 that has been condensed from the reaction zone feed material 22 while the reaction zone feed material 22 is cooled before being supplied to the reaction zone 10, and (b) aqueous material which has been separated from a discharged phototrophic biomass-comprising product 58 (see below). In some embodiments, for example, the supplemental aqueous material supply 44 is derived from an independent source, such as a municipal water supply.

In some embodiments, for example, the supplemental aqueous material supply 44 is supplied by a pump 281. In some of these embodiments, for example, the supplemental aqueous material supply 44 is continuously supplied to the reaction zone 10 which, in some embodiments, effects discharging of at least some of a biomass-comprising product 58 (being discharged from the reaction zone 10, see below) by overflow.

In some embodiments, for example, at least a fraction of the supplemental aqueous material supply 44 is supplied from a container 28, which is further described below. At least a fraction of aqueous material which is discharged from the process is recovered and supplied to the container 28 to provide supplemental aqueous material in the container 28.

Referring to FIG. 3, in some embodiments, the supplemental nutrient supply 42 and the supplemental aqueous material supply 44 are supplied to the reaction zone feed material 22 through the sparger 40 before being supplied to the reaction zone 10. In those embodiments where the reaction zone 10 is disposed in the photobioreactor 12, in some of these embodiments, for example, the sparger 40 is disposed externally of the photobioreactor 12. In some embodiments, it is desirable to mix the reaction zone feed material 22 with the supplemental nutrient supply 42 and the supplemental aqueous material supply 44 within the sparger 40, as this effects better mixing of these components versus separate supplies of the reaction zone feed material 22, the supplemental nutrient supply 42, and the supplemental aqueous material supply 44. On the other hand, the rate of supply of the reaction zone feed material 22 to the reaction zone 10 is limited by virtue of saturation limits of gaseous material of the reaction zone feed material 22 in the combined mixture. Because of this trade-off, such embodiments are more suitable when response time required for providing a modulated supply of carbon dioxide to the reaction zone 10 is not relatively immediate, and this depends on the biological requirements of the phototrophic organisms being used.

In some embodiments, for example, at least a fraction of the supplemental nutrient supply 42 is mixed with the supplemental aqueous material in the container 28 to provide a nutrient-enriched supplemental aqueous material supply 44, and the nutrient-enriched supplemental aqueous material supply 44 is supplied directly to the reaction zone 10 or is mixed with the reaction zone feed material 22 in the sparger 40. In some embodiments, for example, the direct or indirect supply of the nutrient-enriched supplemental aqueous material supply is effected by the pump 281.

The reactor sub-system 120 includes the photobioreactor 12 which is configured for containing the reaction mixture. The reaction mixture defines the respective process material component of the reactor sub-system. The reactor sub-system 120 is configured for exposing the reaction mixture to photosynthetically active light radiation within a reaction zone 10 of a photobioreactor 12 to effect photosynthesis. The photosynthesis effects growth of the phototrophic biomass. The photobioreactor 12 includes at least one inlet for receiving supply material from the material supply sub-system 110, and at least one outlet for discharging reaction zone product 500 from the photobioreactor 12 for supplying the product material processing sub-system 130.

In some embodiments, for example, the photobioreactor 12, or plurality of photobioreactors 12, are configured so as to optimize carbon dioxide absorption by the phototrophic biomass and reduce energy requirements. In this respect, the photobioreactor(s) is (are) configured to provide increased residence time of the carbon dioxide within the reaction zone 10. As well, movement of the carbon dioxide over horizontal distances is minimized, so as to reduce energy consumption. To this end, the photobioreactor 12 is, or are, relatively taller, and provide a reduced footprint, so as to increase carbon dioxide residence time while conserving energy.

The reaction mixture disposed in the reaction zone 10 is exposed to photosynthetically active light radiation so as to effect photosynthesis. The reaction mixture includes the photosynthesis reaction reagents. The photosynthesis effects growth of the phototrophic biomass. In some embodiments, the exposing of the reaction mixture to photosynthetically active light radiation is effected while the supplying of the reaction feed material 22 is being effected.

In some embodiments, for example, photosynthetically active light radiation is characterized by a wavelength of between 400-700 nm.

In some embodiments, for example, the intensity of the provided light is controlled so as to align with the desired growth rate of the phototrophic biomass in the reaction zone 10. In some embodiments, regulation of the intensity of the provided light is based on measurements of the growth rate of the phototrophic biomass in the reaction zone 10. In some embodiments, regulation of the intensity of the provided light is based on the molar rate of supply of carbon dioxide to the reaction zone feed material 22.

In some embodiments, for example, the light is provided at pre-determined wavelengths, depending on the conditions of the reaction zone 10. Having said that, generally, the light is provided in a blue light source to red light source ratio of 1:4. This ratio varies depending on the phototrophic organism being used. As well, this ratio may vary when attempting to simulate daily cycles. For example, to simulate dawn or dusk, more red light is provided, and to simulate mid-day condition, more blue light is provided. Further, this ratio may be varied to simulate artificial recovery cycles by providing more blue light.

It has been found that blue light stimulates algae cells to rebuild internal structures that may become damaged after a period of significant growth, while red light promotes algae growth. Also, it has been found that omitting green light from the spectrum allows algae to continue growing in the reaction zone 10 even beyond what has previously been identified as its “saturation point” in water, so long as sufficient carbon dioxide and, in some embodiments, other nutrients, are supplied.

In some embodiments, for example, the growth rate of the phototrophic biomass is dictated by the available gaseous exhaust material reaction zone supply 24. In turn, this defines the nutrient, water, and light intensity requirements to maximize phototrophic biomass growth rate. In some embodiments, for example, the reactor sub-system 1200 includes a controller, e.g. a computer-implemented system, is provided to be used to monitor and control the operation of the various components of the reactor sub-system 1200, including lights, valves, sensors, blowers, fans, dampers, pumps, etc.

The product material processing sub-system 130 is configured for receiving reaction zone product 500 discharged from the photobioreactor 12 and effecting separation of a liquid component from the received reaction zone product 500. The reaction zone product 500 includes phototrophic biomass-comprising product 58. In this respect, the discharge of the reaction zone product 500 effects harvesting of the phototrophic biomass. In some embodiments, for example, the reaction zone product 500 also includes a reaction zone gaseous effluent product 80. The reaction zone product 500 being discharged defines the respective process material component of the product material processing sub-system 130.

As discussed above, the phototrophic biomass is recovered or harvested by discharging a phototrophic biomass-comprising product 58. The phototrophic biomass-comprising product 58 includes at least a fraction of the contents of the reaction zone 10. With respect to those embodiments where the reaction zone 10 is disposed in a photobioreactor 12, in some of these embodiments, the upper portion of phototrophic biomass suspension in the reaction zone 10 overflows the photobioreactor 12 (for example, the phototrophic biomass is discharged through an overflow port of the photobioreactor 12) to provide the phototrophic biomass-comprising product 58 as an overflow 59. In those embodiments where the phototrophic biomass includes algae, the discharging of the product 58 is effected at a rate which matches the growth rate of the algae, in order to mitigate shocking of the algae in the reaction zone 10. With respect to some embodiments, for example, the discharging of the product 58 is controlled through the rate of supply of supplemental aqueous material supply 44, which influences the displacement from the photobioreactor 12 of the phototrophic biomass-comprising product 58 as an overflow from the photobioreactor 12. In other embodiments, for example, the discharging of the product 58 is controlled with a valve disposed in a fluid passage which is fluidly communicating with an outlet of the photobioreactor 12.

In some embodiments, for example, the discharging of the product is effected continuously. In other embodiments, for example, the discharging of the product is effected periodically. In some embodiments, for example, the discharging of the product is designed such that the concentration of the biomass in the phototrophic biomass-comprising product 58 is maintained at a relatively low concentration. In those embodiments where the phototrophic biomass includes algae, it is desirable, for some embodiments, to effect discharging of the product 58 at lower concentrations to mitigate against sudden changes in the growth rate of the algae in the reaction zone 10. Such sudden changes could effect shocking of the algae, which thereby contributes to lower yield over the longer term. In some embodiments, where the phototrophic biomass is algae and, more specifically, Scenedesmus obliquus, the concentration of these algae in the phototrophic biomass-comprising product 58 could be between 0.5 and 3 grams per litre. The desired concentration of the discharged algae product 58 depends on the strain of algae such that this concentration range changes depending on the strain of algae. In this respect, in some embodiments, maintaining a predetermined water content in the reaction zone is desirable to promote the optimal growth of the phototrophic biomass, and this can also be influenced by controlling the supply of the supplemental aqueous material supply 44.

The phototrophic biomass-comprising product 58 includes water. In some embodiments, for example, the phototrophic biomass-comprising product 58 is supplied to a separator 52 for effecting removal of at least a fraction of the water from the phototrophic biomass-comprising product 58 to effect production of an intermediate concentrated phototrophic biomass-comprising product 34 and a recovered aqueous material 72 (generally, water). In some embodiments, for example, the separator 52 is a high speed centrifugal separator 52. Other suitable examples of a separator 52 include a decanter, a settling vessel or pond, a flocculation device, or a flotation device. In some embodiments, the recovered aqueous material 72 is supplied to a container 28, such as a container, for re-use by the process.

In some embodiments, for example, after the product 58 is discharged, and before being supplied to the separator 52, the phototrophic biomass-comprising product 58 is supplied to a harvest pond 54. The harvest pond 54 functions both as a buffer between the photobioreactor 12 and the separator 52, as well as a mixing vessel in cases where the harvest pond 54 receives different biomass strains from multiple photobioreactors. In the latter case, customization of a blend of biomass strains can be effected with a predetermined set of characteristics tailored to the fuel type or grade that will be produced from the blend.

As described above, the container 28 provides a source of supplemental aqueous material supply 44 for the reaction zone 10. Loss of water is experienced in some embodiments as moisture in the final phototrophic biomass-comprising product 36, as well as through evaporation in the dryer 32. The supplemental aqueous material in the container 28, which is recovered from the process, can be supplied to the reaction zone 10 as the supplemental aqueous material supply 44. In some embodiments, for example, the supplemental aqueous material supply 44 is supplied to the reaction zone 10 with a pump. In other embodiments, the supply can be effected by gravity, if the layout of the process equipment of the system, which embodies the process, permits. As described above, the supplemental aqueous material recovered from the process includes at least one of: (a) aqueous material 70 which has been condensed from the reaction zone feed material 22 while the reaction zone feed material 22 is being cooled before being supplied to the reaction zone 10, and (b) aqueous material 72 which has been separated from the phototrophic biomass-comprising product 58. In some embodiments, for example, the supplemental aqueous material supply 44 is supplied to the reaction zone 10 to influence overflow of the product 58 by increasing the upper level of the contents of the reaction zone 10. In some embodiments, for example, the supplemental aqueous material supply 44 is supplied to the reaction zone 10 to influence a desired predetermined concentration of phototrophic biomass to the reaction zone by diluting the contents of the reaction zone.

Examples of specific structures which can be used as the container 28 by allowing for containment of aqueous material recovered from the process, as above-described, include, without limitation, tanks, ponds, troughs, ditches, pools, pipes, tubes, canals, and channels.

In some embodiments, for example, the supplying of the supplemental aqueous material supply to the reaction zone 10 is effected while the gaseous exhaust material 18 is being produced by the gaseous exhaust material producing process 20. In some embodiments, for example, the supplying of the supplemental aqueous material supply to the reaction zone is effected while the gaseous exhaust material reaction zone supply 24 is being supplied to the reaction zone feed material 22. In some embodiments, for example, the supplying of the supplemental aqueous material supply to the reaction zone 10 is effected while the reaction zone feed material 22 is being supplied to the reaction zone 10. In some embodiments, for example, the exposing of the carbon dioxide-enriched phototrophic biomass disposed in the aqueous medium to photosynthetically active light radiation is effected while the supplying of the supplemental aqueous material supply to the reaction zone 10 is being effected.

In some embodiments, for example, when the upper level of the contents of the reaction zone 10 within the photobioreactor 12 becomes disposed below a predetermined minimum level, the initiation of the supply of, or an increase to the molar rate of supply of, the supplemental aqueous material supply 44 (which has been recovered from the process) is effected to the reaction zone 10. In some of these embodiments, for example, a level sensor 76 is provided for sensing the position of the upper level of the contents of the reaction zone 10 within the photobioreactor, and transmitting a signal representative of the upper level of the contents of the reaction zone 10 to the controller. Upon the controller comparing a received signal from the level sensor 76, which is representative of the upper level of the contents of the reaction zone 10, to a predetermined low level value, and determining that the sensed upper level of the contents of the reaction zone is below the predetermined low level value, the controller effects the initiation of the supply of, or an increase to the molar rate of supply of, the supplemental aqueous material supply 44. When the supply of the supplemental aqueous material supply 44 to the reaction zone 10 is effected by a pump 281, the controller actuates the pump 281 to effect the initiation of the supply of, or an increase to the rate of supply of, the supplemental aqueous material supply 44 to the reaction zone 10. When the supply of the supplemental aqueous material supply 44 to the reaction zone 10 is effected by gravity, the controller actuates the opening of a control valve to effect the initiation of the supply, or an increase to the molar rate of supply of, the supplemental aqueous material supply 44 to the reaction zone 10. For example, control of the position of the upper level of the contents of the reaction zone 10 is relevant to operation for some of those embodiments where harvesting is effected from a lower portion of the reaction zone 10. In those embodiments where harvesting is effected by an overflow, in some of these embodiments, control of the position of the upper level of the contents of the reaction zone 10 is relevant during the “seeding stage” of operation of the photobioreactor 12.

In some embodiments, supply of the supplemental aqueous material supply 44 to the reaction zone 10 is dictated by algae concentration. In this respect, molar algae concentration in the reaction zone is sensed by a cell counter, such as the cell counters described above. The sensed molar algae concentration is transmitted to the controller, and when the controller determines that the sensed molar algae concentration exceeds a predetermined high algae concentration value, and when the supply of the supplemental aqueous material supply 44 to the reaction zone 10 is effected by a pump 281, the controller responds by actuating the pump 281 to effect supply of the supplemental aqueous material supply 44 to the reaction zone 10. When the supply of the supplemental aqueous material supply 44 to the reaction zone 10 is effected by gravity, the controller actuates the opening of a control valve to effect the initiation of the supply, or an increase to the molar rate of supply of, the supplemental aqueous material supply 44 to the reaction zone 10.

In some embodiments, for example, where the discharging of the product 58 is controlled with a valve 441 disposed in a fluid passage which is fluidly communicating with an outlet of the photobioreactor 12, molar concentration of algae in the reaction zone is sensed by a cell counter, such as the cell counters described above. The sensed molar concentration of algae is transmitted to the controller, and when the controller determines that the sensed molar algae concentration exceeds a predetermined high molar algae concentration value, the controller responds by actuating opening of the valve to effect an increase in the molar rate of discharging of the product 58 from the reaction zone 10.

In some embodiments, for example, a source of additional make-up water 68 is provided to mitigate against circumstances when the supplemental aqueous material supply 44 is insufficient to make-up for water which is lost during operation of the process. In this respect, in some embodiments, for example, the supplemental aqueous material supply 44 is mixed with the reaction zone feed material 22 in the sparger 40. Conversely, in some embodiments, for example, accommodation for draining of the container 28 to drain 66 is provided to mitigate against the circumstances when aqueous material recovered from the process exceeds the make-up requirements.

The intermediate concentrated phototrophic biomass-comprising product 34 is supplied to a dryer 32 which supplies heat to the intermediate concentrated phototrophic biomass-comprising product 34 to effect evaporation of at least a fraction of the water of the intermediate concentrated phototrophic biomass-comprising product 34, and thereby effect production of a final phototrophic biomass-comprising product 36. As discussed above, in some embodiments, the heat supplied to the intermediate concentrated phototrophic biomass-comprising product 34 is provided by a heat transfer medium 30 which has been used to effect the cooling of the reaction zone feed material 22 prior to supply of the reaction zone feed material 22 to the reaction zone 10. By effecting such cooling, heat is transferred from the reaction zone feed material 22 to the heat transfer medium 30, thereby raising the temperature of the heat transfer medium 30. In such embodiments, the intermediate concentrated phototrophic biomass-comprising product 34 is at a relatively warm temperature, and the heat requirement to effect evaporation of water from the intermediate concentrated phototrophic biomass-comprising product 34 is not significant, thereby rendering it feasible to use the heated heat transfer medium 30 as a source of heat to effect the drying of the intermediate concentrated phototrophic biomass-comprising product 34. As discussed above, after heating the intermediate concentrated phototrophic biomass-comprising product 34, the heat transfer medium 30, having lost some energy and becoming disposed at a lower temperature, is recirculated to the heat exchanger 26 to effect cooling of the reaction zone feed material 22. The heating requirements of the dryer 32 are based upon the rate of supply of intermediate concentrated phototrophic biomass-comprising product 34 to the dryer 32. Cooling requirements (of the heat exchanger 26) and heating requirements (of the dryer 32) are adjusted by the controller to balance the two operations by monitoring flow rates and temperatures of each of the reaction zone feed material 22 and the rate of production of the product 58 through discharging of the product 58 from the photobioreactor.

In some embodiments, changes to the phototrophic biomass growth rate effected by changes to the rate of supply of the gaseous exhaust material reaction zone supply 24 to the reaction zone material feed 22 are realized after a significant time lag (for example, in some cases, more than three (3) hours, and sometimes even longer) from the time when the change is effected to the rate of supply of the gaseous exhaust material reaction zone supply 24 to the reaction zone feed material 22. In comparison, changes to the thermal value of the heat transfer medium 30, which are based on the changes in the rate of supply of the gaseous exhaust material reaction zone supply 24 to the reaction zone feed material 22, are realized more quickly. In this respect, in some embodiments, a thermal buffer is provided for storing any excess heat (in the form of the heat transfer medium 30) and introducing a time lag to the response of the heat transfer characteristics of the dryer 32 to the changes in the gaseous exhaust material reaction zone supply 24. In some embodiments, for example, the thermal buffer is a heat transfer medium storage tank. Alternatively, an external source of heat may be required to supplement heating requirements of the dryer 32 during transient periods of supply of the gaseous exhaust material reaction zone supply 24 to the reaction zone material 22. The use of a thermal buffer or additional heat may be required to accommodate changes to the rate of growth of the phototrophic biomass, or to accommodate start-up or shutdown of the process. For example, if growth of the phototrophic biomass is decreased or stopped, the dryer 32 can continue operating by using the stored heat in the buffer until it is consumed, or, in some embodiments, use a secondary source of heat.

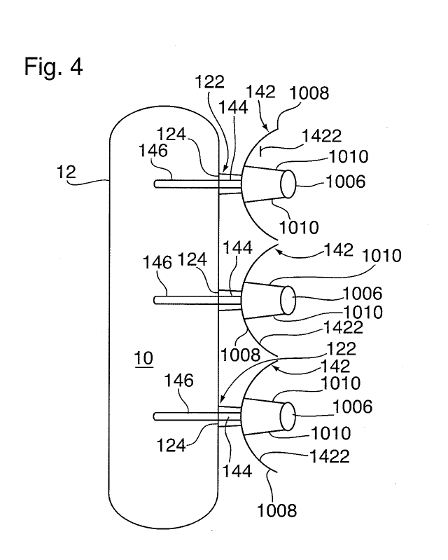

Referring to FIGS. 4 to 7, the solar energy supply sub-system 140 includes at least one solar collector 142, and each one of the at least one solar collector 142 includes a solar collector surface configured for receiving incident solar radiation.

Examples of suitable solar collectors 142 include parabolic dish collectors, Fresnel lens, and a Cassegrain optical system.

Each one of the at least one solar collector 142 is operatively coupled to an energy supply component 144 configured for supplying energy derived from the received incident solar radiation and supplying the energy to at least one of the other sub-systems 110, 120, or 130.

In some embodiments, for example, for at least one of the at least one solar collector 142, the energy supply component 144, to which the solar collector 142 is operatively coupled, includes a light transmission component 146 configured for supplying light energy, derived from at least a fraction of the received incident solar radiation, to the reaction mixture disposed within the reaction zone 10 of the photobioreactor 12 to thereby effect exposure of the reaction mixture to photosynthetically active light radiation. In some embodiments, for example, the solar collector 142 is operatively coupled to the light transmission component 146 with an optical fibre. Exemplary light transmission components include waveguides, light guides, liquid light guides, light tubes, and optical fibres.

In some embodiments, for example, the light transmission component 146 includes one or more optical waveguides (or “light guides”). In this respect, each one of the one or more optical waveguides is operatively coupled to the solar collector surface with an optical fiber to effect transmission of at least a fraction of the received incident solar radiation for supplying to the reaction mixture disposed within the reaction zone 10 of the photobioreactor 12 to thereby effect exposure of the reaction mixture to photosynthetically active light radiation.

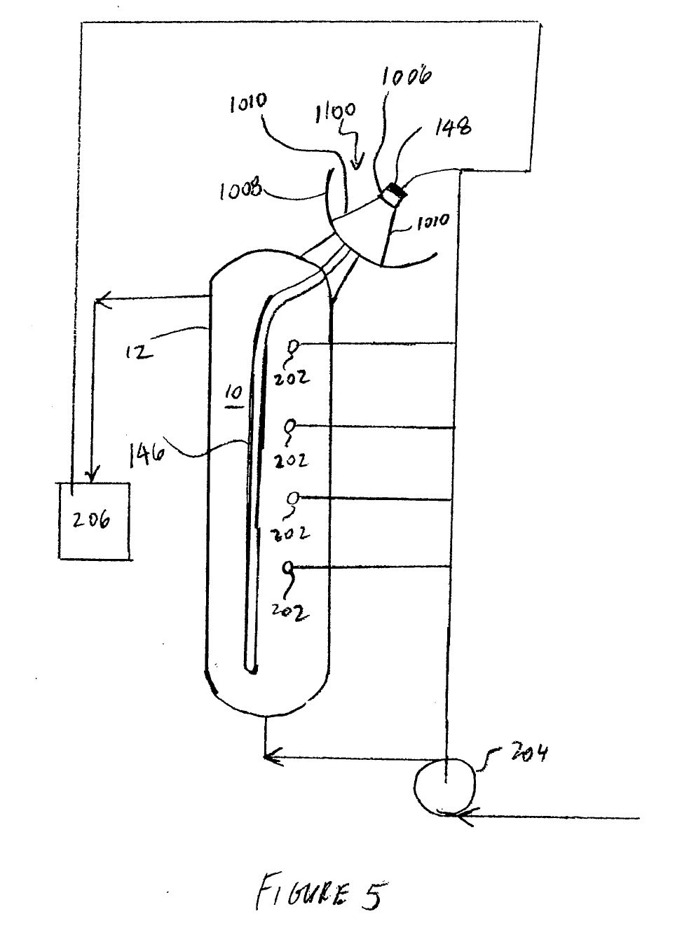

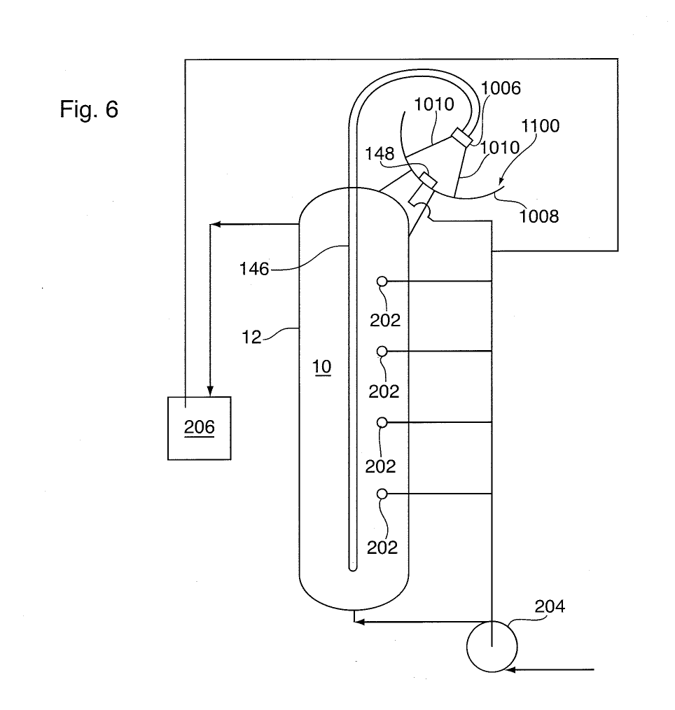

In some embodiments, for example, and referring to FIGS. 5 and 6, the solar energy supply sub-system 140 includes a filtering component 148 for at least one of the at least one solar collector 142. The filtering component 148 is configured to filter the received incident solar radiation and effects the provision of light of desired wavelengths to the reaction zone 10. For example, filtering includes filtering with custom mirrors. In this respect, the filtered light is provided by the solar collector 142 to the light transmission component 146.

In some embodiments, for example, in addition to the light derived from the received incident solar radiation, the light transmission component 146 includes high power LED arrays that can provide light at specific wavelengths to either complement solar light, as necessary, or to provide all of the necessary light to the reaction zone 10 during periods of darkness (for example, at night). In some embodiments, with respect to the light guides, for example, a transparent heat transfer medium (such as a glycol solution) is circulated through light guides within the photobioreactor 12 so as to regulate the temperature in the light guides and, in some circumstances, provide for the controlled dissipation of heat from the light guides and into the reaction zone 10. In some embodiments, for example, the LED power requirements can be predicted and, therefore, controlled, based on trends observed with respect to the gaseous exhaust material 18, as these observed trends assist in predicting future growth rate of the phototrophic biomass.

In some embodiments, for example, and referring to FIGS. 5 and 6, for at least one of the at least one solar collector 142, the energy supply component 144, to which the solar collector 142 is operatively coupled, includes an energy conversion and supply component 148 configured for converting at least a fraction of the received incident solar radiation to electricity and transmitting the electricity for powering one or more electrical loads, each of which is configured for supplying energy to another at least one of the sub-systems 110, 120, or 130. In some embodiments, for example, the supplying energy to another at least one of the sub-systems includes effecting the supply of energy to a respective process material component of another at least one of the sub-systems 110, 120, or 130. In some embodiments, for example, the energy conversion and supply component 148 includes a photovoltaic cell that is electrically coupled to the one or more electrical loads.

Alternatively, in some embodiments, the energy conversion and supply component 148 is configured for converting at least a fraction of the received incident solar radiation to thermal energy and supplying the thermal energy to one or more heat sinks of another at least one of the sub-systems 110, 120, or 130. In this respect, in some embodiments, for example, the energy conversion and supply component includes a solar heater for absorbing the incident solar radiation and converting the received incident solar radiation into thermal energy of a working fluid of the solar heater. The heated working fluid is then supplied to various heat sinks provided in another at least one of the sub-systems 110, 120, or 130, such as the dryer 32.

In some embodiments, for example, the electrical load being powered by electricity being supplied by the energy conversion and supply component 148 is an artificial light source 202 configured for supplying photosynthetically active light radiation to the reaction zone 10 and thereby effecting exposure of the reaction mixture to the photosynthetically active light radiation. With respect to artificial light sources, for example, suitable artificial light sources include submersible fibre optics, light-emitting diodes (“LEDs,” including submersible LEDs), LED strips, and fluorescent lights. In some embodiments, for example, the light transmission component 146 includes one or more artificial light sources 202.

In some embodiments, for example, the electrical load being powered by electricity being supplied by the energy conversion and supply component is an electrical load of the reactor sub-system 120, such as any one of lights, valves, sensors, controllers, blowers, fans, dampers, and pumps of the reactor sub-system 120.

In some embodiments, for example, the electrical load being powered by electricity being supplied by the energy conversion and supply component is an electrical load of the product material processing sub-system 130, such as any one of lights, valves, sensors, controllers, blowers, fans, dampers, and pumps of the product material processing sub-system 130.

In some embodiments, for example, and referring to FIGS. 5 and 6, the solar energy supply sub-system 140 includes a filter/mirror assembly 1006 (including, for example, any one of an interference filter/mirror assembly, a dielectric elliptical mirror, or a dichromic mirror filter) that filters the incident solar radiation received by the solar collector to provide a light source-purpose received incident solar radiation fraction and a power generation-purpose received incident solar radiation fraction. The light source-purpose received incident solar radiation fraction is of desirable wavelengths for purposes of effecting photosynthesis upon its exposure to the reaction mixture within the reaction zone 10. In some embodiments, for example, the light source-purpose received incident solar radiation fraction is of a light of a wavelength between about 400-700 nm. In some embodiments, for example, the light source-purpose received incident solar radiation fraction is visible light. The light source-purpose received incident solar radiation fraction is transmitted to the reaction zone with the light transmission component 146. The power generation-purpose received incident solar radiation fraction is transmitted to the energy conversion and supply component, for conversion to electricity for powering one or more of the electrical loads, or for conversion to thermal energy for transferring to a heat sink.

Referring to FIG. 5, in some embodiments, for example, the solar collector 142 includes a reflective, parabolic dish 1008. The interference filter/mirror assembly 1006 is mounted to and supported by the dish 1008 with supports 1010. The photovoltaic cell 148 is mounted behind the interference filter/mirror assembly 1006 for receiving the power generation-purpose received incident solar radiation fraction. The parabolic dish 1008 is configured to reflect (with parabolic focus) incident solar radiation that impinges on the dish 1008 onto the interference filter/mirror assembly 1006. In some embodiments, for example, the interference filter/mirror assembly 1006 is a cold mirror which reflects visible light while transmitting infrared light. The interference filter/mirror assembly 1006 is configured to reflect, focus, and concentrate the light source-purpose received incident solar radiation fraction of the incident solar radiation (reflected by the dish 1008) onto a light transmission component 146, which transmits the received light source-purpose received incident solar radiation fraction to the reaction zone 10. The interference filter/mirror assembly 1006 is also configured to permit the transmission of the power generation-purpose received incident solar radiation fraction through to the photovoltaic cell 148. The photovoltaic cell 148 converts the received power generation-purpose received incident solar radiation fraction to electricity for powering one or more of the electrical loads of another at least one of the sub-systems 110, 120, or 130. For example, the one or more electrical loads include LED lighting 202. For example, the one or more of the electrical loads include a prime mover 204 (such as a blower or a fan) for supplying the carbon comprising gas to the reaction zone. For example, the one or more of the electrical loads include a dryer 206 for effecting drying of phototrophic biomass-comprising product recovered from the reaction zone 10.

Another configuration is illustrated in FIG. 6. In this configuration, the light transmission component 146 extends from behind the interference filter/mirror assembly 1006 and the photovoltaic cell 148 is positioned relative to the dish 1008 to receive focussed and concentrated power generation-purpose received incident solar radiation fraction that is being reflected from the interference filter/mirror assembly 1006.

In another aspect, and referring to FIGS. 4 to 7, at least one of the at least one solar collector 142 is mounted to the photobioreactor 12, such that at least one photobioreactor-mounted solar collector surface 1422 is provided to define a total photobioreactor-mounted solar collector surface area, wherein the total photobioreactor-connected solar collector surface area is at least 50 square metres. In some embodiments, for example, the total photobioreactor-connected solar collector surface area is at least 75 square metres. In some embodiments, for example, the total photobioreactor-connected solar collector surface area is at least 100 square metres. In some embodiments, for example, the total photobioreactor-connected solar collector surface area is at least 150 square metres. In some embodiments, for example, the total photobioreactor-connected solar collector surface area is at least 250 square metres and the plurality of solar collectors defines the total photobioreactor-connected solar collector surface area.

In another aspect, and referring to FIG. 4, the solar energy supply sub-system 140 includes at least one solar collector 142, and each one of the at least one solar collector 142 is mounted to an operative mounting surface 122 of the photobioreactor 12 and includes a solar collector surface 1422 configured for receiving incident solar radiation. In some embodiments, for example, the at least one solar collector is a plurality of solar collectors. The operative mounting surface is oriented within 45 degrees of the vertical. In some embodiments, for example, the operative mounting surface is oriented within 25 degrees of the vertical. In some embodiments, for example, the operative mounting surface is oriented within 15 degrees of the vertical. In some embodiments, for example, the operative mounting surface is oriented within 5 degrees of the vertical. In some embodiments, for example, the photobioreactor system is disposed either at least 25 degrees north of the equator or at least 25 degrees south of the equator. In some embodiments, for example, each one of the at least one solar collector is mounted to the photobioreactor 12 and includes a solar collector surface configured for receiving incident solar radiation such that at least one solar collector surface is provided to define a total photobioreactor-connected solar collector surface area, wherein the total photobioreactor-connected solar collector surface area is at least 50 square metres. In some embodiments, for example, the total photobioreactor-connected solar collector surface area is at least 75 square metres. In some embodiments, for example, the total photobioreactor-connected solar collector surface area is at least 100 square metres. In some embodiments, for example, the total photobioreactor-connected solar collector surface area is at least 150 square metres. In some embodiments, for example, the total photobioreactor-connected solar collector surface area is at least 250 square metres. In some embodiments, for example, the at least one solar collector is a plurality of solar collectors, and the plurality of solar collectors defines the total photobioreactor-connected solar collector surface area. In some embodiments, for example, the solar collector 142 includes a reflective, parabolic dish 1008. In some embodiments, for example, an interference filter/mirror assembly 1006 is mounted to and supported by the dish 1008 with supports 1010. The parabolic dish 1008 is configured to reflect (with parabolic focus) incident solar radiation that impinges on the dish 1008 onto the interference filter/mirror assembly 1006. The interference filter/mirror assembly 1006 filters the incident solar radiation received by the solar collector to provide a light source-purpose received incident solar radiation fraction. The interference filter/mirror assembly 1006 is configured to reflect, focus, and concentrate the light source-purpose received incident solar radiation fraction of the incident solar radiation (reflected by the dish 1008) onto the light transmission component 146, which transmits the received light source-purpose received incident solar radiation fraction to the reaction zone 10, while filtering out the incident solar radiation (reflected by the dish 1008) that is of certain wavelengths (e.g., infrared) which are not useful, or substantially not useful, for purposes of effecting photosynthesis upon its exposure to the reaction mixture within the reaction zone 10, and thereby mitigate heating of the reaction zone by light which is not useful, or substantially not useful, for purposes of effecting photosynthesis upon its exposure to the reaction mixture within the reaction zone 10. In some embodiments, for example, the interference filter/mirror assembly 1006 is a cold mirror which reflects visible light while transmitting infrared light. In some embodiments, as a result of the filtering, a power generation-purpose received incident solar radiation fraction is also provided. In some embodiments, where, as a result of the filtering, a power generation-purpose received incident solar radiation fraction is also provided, a photovoltaic cell is mounted behind the interference filter/mirror assembly 1006 for receiving the power generation-purpose received incident solar radiation fraction. The photovoltaic cell converts the received power generation-purpose received incident solar radiation fraction to electricity for powering one or more of the electrical loads of another at least one of the sub-systems 110, 120, or 130.

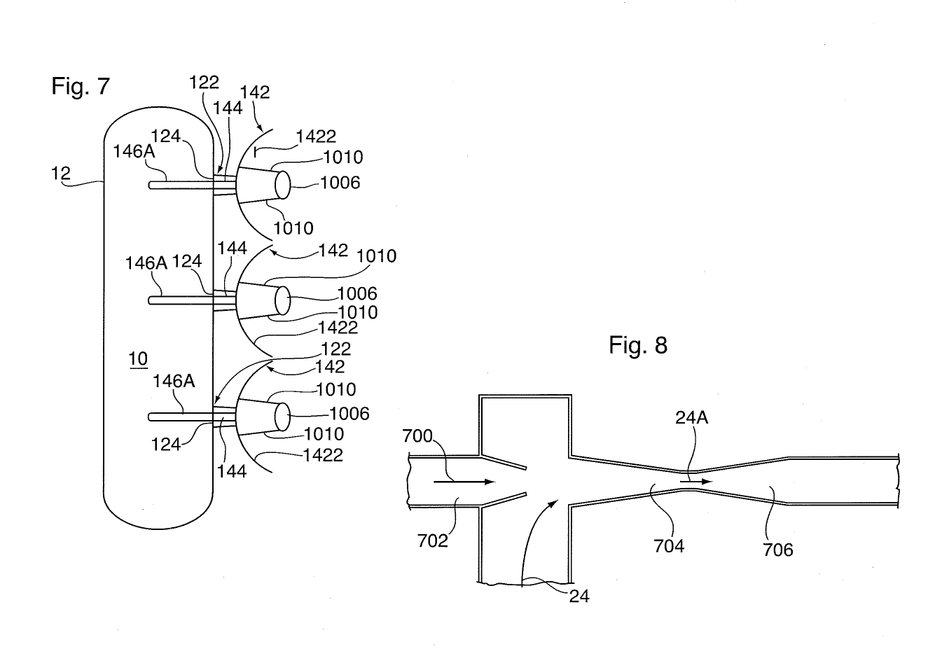

In another aspect, and referring to FIG. 7, a plurality of operative light transmission components 146A are provided and are configured for supplying light energy to the reaction zone 10 of the photobioreactor 12 to thereby effect exposure of the reaction mixture within at least 80% of the reaction zone to photosynthetically active light radiation. In some embodiments, for example, the plurality of operative light transmission components 146A, which are provided, are configured for supplying light energy to the reaction zone 10 of the photobioreactor 12 to thereby effect exposure of the reaction mixture within at least 90% of the reaction zone to photosynthetically active light radiation. Each one of the operative light transmission components 146A is mounted to and extends into the reaction zone 10 (defined within the photobioreactor 12) from an operative portion 124 of an internal surface of the photobioreactor 12 for an operative distance, wherein the volume of the reaction zone is greater than 3000 litres. In some embodiments, the volume of the reaction zone is greater than 5000 litres. The operative distance is less than five (5) metres. In some embodiments, for example, the operative distance is less than three (3) metres. Exemplary light transmission components include waveguides, light guides, liquid light guides, light tubes, and optical fibres. In some embodiments, the operative light transmission component 146A includes an artificial light source, such as LEDs. In this respect, in some embodiments, for example, the operative light transmission components 146A includes LEDs for effecting supply of the light energy to the reaction zone 10. In some embodiments, for example, at least a fraction of the light energy being transmitted by the operative light transmission components 146A is derived from the incident solar radiation received by at least one solar collector 142. In some embodiments, for example, the solar collector 142 includes a reflective, parabolic dish 1008 for receiving incident solar radiation and supplying at least a fraction of the received incident solar radiation to the light transmission component 146A. In some embodiments, for example, an interference filter/mirror assembly 1006 is mounted to and supported by the dish 1008 with supports 1010. The parabolic dish 1008 is configured to reflect (with parabolic focus) incident solar radiation that impinges on the dish 1008 onto the interference filter/mirror assembly 1006. The interference filter/mirror assembly 1006 filters the incident solar radiation received by the solar collector to provide a light source-purpose received incident solar radiation fraction. The interference filter/mirror assembly 1006 is configured to reflect, focus, and concentrate the light source-purpose received incident solar radiation fraction of the incident solar radiation (reflected by the dish 1008) onto the light transmission component 146A, which transmits the received light source-purpose received incident solar radiation fraction to the reaction zone 10, while filtering out the incident solar radiation (reflected by the dish 1008) that is of certain wavelengths (e.g., infrared) which are not useful, or substantially not useful, for purposes of effecting photosynthesis upon its exposure to the reaction mixture within the reaction zone 10, and thereby mitigate heating of the reaction zone by light which is not useful, or substantially not useful, for purposes of effecting photosynthesis upon its exposure to the reaction mixture within the reaction zone 10. In some embodiments, for example, the interference filter/mirror assembly 1006 is a cold mirror which reflects visible light while transmitting infrared light. In some embodiments, as a result of the filtering, a power generation-purpose received incident solar radiation fraction is also provided. In some embodiments, where, as a result of the filtering, a power generation-purpose received incident solar radiation fraction is also provided, a photovoltaic cell is mounted behind the interference filter/mirror assembly 1006 for receiving the power generation-purpose received incident solar radiation fraction. The photovoltaic cell converts the received power generation-purpose received incident solar radiation fraction to electricity for powering one or more of the electrical loads of another at least one of the sub-systems 110, 120, or 130.

In another aspect, and referring to FIGS. 4 and 7, the solar energy supply sub-system 140 includes a plurality of solar collectors. Each one of the plurality of solar collectors includes a solar collector surface configured for receiving incident solar radiation. The solar energy supply sub-system also includes a plurality of vertically spaced energy supply components (in this case, light transmission components 146 or 146A). Each one of the vertically spaced energy supply components is configured for transmitting energy derived from the received incident solar radiation and supplying the energy to at least one of the other sub-systems 110, 120, or 130. Each one of the plurality of solar collectors is operatively coupled to at least one of the vertically spaced energy supply components. Each one of the vertically spaced energy supply components extends into the reaction zone 10 from an operative portion of an internal surface of the photobioreactor 12. The internal surface of the photobioreactor 12 defines a space including the reaction zone 10. Each one of the vertically spaced energy supply components is disposed at a different vertical position relative to the other ones of the vertically spaced energy supply components. In some embodiments, for example, for at least two of the energy supply components, the energy supply component includes a light transmission component configured for transmitting light energy, derived from at least a fraction of the received incident solar radiation, and effecting its supply to the reaction mixture disposed within the reaction zone 10 of the photobioreactor 12 to thereby effect exposure of the reaction mixture to photosynthetically active light radiation.

In some embodiments, for example, each one of the vertically spaced energy transmission devices extends into the reaction zone from an operative portion of an internal surface, such that a plurality of internal operative surface portions are provided. Each one of the internal operative surface portions is disposed at a different vertical position relative to the other ones of the internal operative surface portions. In some embodiments, for example, each one of the internal operative surface portions is oriented within 45 degrees of the vertical. In some embodiments, for example, each one of the vertically spaced energy supply components is mounted to the photobioreactor In some embodiments, for example, for at least one of the at least one solar collector, at least one of the at least one energy supply component, to which the solar collector is operatively coupled, includes a light transmission component configured for transmitting light energy, derived from at least a fraction of the received incident solar radiation, and effecting its supply to the reaction mixture disposed within the reaction zone 10 of the photobioreactor 12 to thereby effect exposure of the reaction mixture to photosynthetically active light radiation. In some embodiments, for example, the photobioreactor 12 includes a reaction zone 10 including a minimum vertical extent that is greater than the diameter or the width of the reaction zone 10.

In the above description, for purposes of explanation, numerous details are set forth in order to provide a thorough understanding of the present disclosure. However, it will be apparent to one skilled in the art that these specific details are not required in order to practice the present disclosure. Although certain dimensions and materials are described for implementing the disclosed example embodiments, other suitable dimensions and/or materials may be used within the scope of this disclosure. All such modifications and variations, including all suitable current and future changes in technology, are believed to be within the sphere and scope of the present disclosure. All references mentioned are hereby incorporated by reference in their entirety.

What is claimed is:

1. A photobioreactor system comprising:
a supply material processing sub-system;
a reactor sub-system including a photobioreactor configured for containing a reaction mixture that is operative for effecting photosynthesis upon exposure to photosynthetically active light radiation, wherein the reaction mixture includes photosynthesis reaction reagents;
wherein the supply material processing sub-system is configured for supplying the reactor with supply material, wherein the supply material includes at least one of the photosynthesis reaction reagents;
a product material processing sub-system configured for receiving reaction zone product discharged from the reactor and effecting separation of a liquid component from the received reaction zone product; and
a solar energy supply sub-system including at least one solar collector, wherein each one of the at least one solar collector is mounted to the photobioreactor and includes a solar collector surface configured for receiving incident solar radiation such that at least one solar collector surface is provided to define a total photobioreactor-connected solar collector surface area, wherein each one of the at least one solar collector is operatively coupled to an energy supply component that is configured for transmitting energy derived from the received incident solar radiation and supplying the energy to at least one of the other sub-systems;
wherein the total photobioreactor-connected solar collector surface area is at least 75 square metres.

2. The photobioreactor system as claimed in claim 1, wherein the photosynthesis reaction reagents include phototrophic biomass material, carbon dioxide, and water.

3. The photobioreactor as claimed in claim 1, wherein for at least one of the at least one solar collector, the energy supply component includes an energy conversion and supply component configured for converting at least a fraction of the received incident solar radiation to electricity and transmitting the electricity for powering one or more electrical loads, each of which is configured for supplying energy to at least one of the other sub-systems.

4. The photobioreactor as claimed in claim 3,
wherein the supplying energy to at least one of the other sub-systems includes effecting the supply of energy to a respective process material component of at least one of the other sub-systems;
and wherein the photosynthesis reaction reagents being supplied by the supply material processing sub-system define the respective process material component of the supply material processing sub-system;
and wherein the photosynthesis reaction reagents within the reaction zone define the respective process material component of the reactor sub-system;
and wherein the reaction zone product discharged from the photobioreactor defines the respective process material component of the product material processing sub-system.

5. The photobioreactor as claimed in claim 3, wherein, for at least one of the at least one solar collector, the respective energy conversion and supply component includes a photovoltaic cell that is electrically coupled to the one or more electrical loads.

6. The photobioreactor as claimed in claim 1, wherein, for at least one of the at least one solar collector, the energy supply component, to which the solar collector is operatively coupled, includes a light transmission component configured for transmitting light energy, derived from at least a fraction of the received incident solar radiation, and effecting its supply to the reaction mixture disposed within the reaction zone of the photobioreactor to thereby effect exposure of the reaction mixture to photosynthetically active light radiation.

7. A photobioreactor system comprising:
a supply material processing sub-system;
a reactor sub-system including a photobioreactor configured for containing a reaction mixture that is operative for effecting photosynthesis upon exposure to photosynthetically active light radiation, wherein the reaction mixture includes photosynthesis reaction reagents;
wherein the supply material processing sub-system is configured for supplying the reactor with supply material, wherein the supply material includes at least one of the photosynthesis reaction reagents;
a product material processing sub-system configured for receiving reaction zone product discharged from the reactor and effecting separation of a liquid component from the received reaction zone product; and
a solar energy supply sub-system including at least one solar collector, wherein each one of the at least one solar collector is mounted to an operative mounting surface of the photobioreactor and includes a solar collector surface configured for receiving incident solar radiation, wherein each one of the at least one solar collector is operatively coupled to an energy supply component that is configured for transmitting energy derived from the received incident solar radiation and supplying the energy to at least one of the other sub-systems;
wherein the operative mounting surface is oriented within 45 degrees of the vertical.

8. The photobioreactor system as claimed in claim 7, wherein the operative mounting surface is oriented within 25 degrees of the vertical.

9. The photobioreactor system as claimed in claim 7, wherein the operative mounting surface is oriented within 15 degrees of the vertical.

10. The photobioreactor system as claimed in claim 7, wherein the operative mounting surface is oriented within 5 degrees of the vertical.

11. The photobioreactor system as claimed in claim 7, wherein the photobioreactor system is disposed either at least 25 degrees north of the equator or at least 25 degrees south of the equator.

12. The photobioreactor as claimed in claim 7, wherein, for at least one of the at least one solar collector, the energy supply component, to which the solar collector is operatively coupled, includes a light transmission component configured for transmitting light energy, derived from at least a fraction of the received incident solar radiation, and effecting its supply to the reaction mixture disposed within the reaction zone of the photobioreactor to thereby effect exposure of the reaction mixture to photosynthetically active light radiation.

13. The photobioreactor system as claimed in claim 7, wherein each one of the at least one solar collector is mounted to the photobioreactor and includes a solar collector surface configured for receiving incident solar radiation such that at least one solar collector surface is provided to define a total photobioreactor-connected solar collector surface area, wherein the total photobioreactor-connected solar collector surface area is at least 75 square metres.

14. A photobioreactor system comprising:
a photobioreactor configured for containing a reaction mixture in a reaction zone, wherein the reaction mixture is operative for effecting photosynthesis upon exposure to photosynthetically active light radiation, and includes photosynthesis reaction reagents, and wherein the reaction zone includes a volume of at least 3000 litres;
a plurality of operative light transmission components configured for supplying light energy to the reaction zone of the photobioreactor to thereby effect exposure of the reaction mixture within at least 80% of the reaction zone to photosynthetically active light radiation, wherein each one of the operative light transmission components is mounted to and extends into the reaction zone from an operative portion of an internal surface of the photobioreactor for an operative distance, wherein the operative distance is less than five (5) metres.

15. The photobioreactor system as claimed in claim 14, wherein at least a fraction of the light energy which the light transmission components are configured to supply is derivable from incident solar radiation received by a solar collector mounted to the photobioreactor.

16. A photobioreactor system comprising:
a supply material processing sub-system;
a reactor sub-system including a photobioreactor configured for containing a reaction mixture that is operative for effecting photosynthesis upon exposure to photosynthetically active light radiation, wherein the reaction mixture includes photosynthesis reaction reagents;
wherein the supply material processing sub-system is configured for supplying the reactor with supply material, wherein the supply material includes at least one of the photosynthesis reaction reagents;
a product material processing sub-system configured for receiving reaction zone product discharged from the reactor and effecting separation of a liquid component from the received reaction zone product; and
a solar energy supply sub-system including at least one solar collector, wherein each one of the at least one solar collector includes a solar collector surface configured for receiving incident solar radiation, and also including a plurality of vertically spaced energy supply components, wherein each one of the vertically spaced energy supply components is configured for transmitting energy derived from the received incident solar radiation and supplying the energy to at least one of the other sub-systems, wherein each one of the at least one solar collector is operatively coupled to at least one of the vertically spaced energy supply components;
wherein each one of the vertically spaced energy supply components extends into the reaction zone from an operative portion of an internal surface of the photobioreactor, wherein the internal surface of the photobioreactor defines a space including the reaction zone;
wherein each one of the vertically spaced energy supply components is disposed at a different vertical position relative to the other ones of the vertically spaced energy supply components.

17. The photobioreactor system as claimed in claim 16, wherein each one of the vertically spaced energy transmission devices extends into the reaction zone from an operative portion of an internal surface, such that a plurality of internal operative surface portions are provided, wherein each one of the internal operative surface portions is disposed at a different vertical position relative to the other ones of the internal operative surface portions.

18. The photobioreactor system as claimed in claim 16, wherein each one of the internal operative surface portions is oriented within 45 degrees of the vertical.

19. The photobioreactor system as claimed in claim 16, wherein each one of the vertically spaced energy transmission devices is mounted to the photobioreactor.

20. The photobioreactor system as claimed in claim 16, wherein, for at least one of the at least one solar collector, at least one of the at least one energy supply component, to which the solar collector is operatively coupled, includes a light transmission component configured for transmitting light energy, derived from at least a fraction of the received incident solar radiation, and effecting its supply to the reaction mixture disposed within the reaction zone of the photobioreactor to thereby effect exposure of the reaction mixture to photosynthetically active light radiation.

21. The photobioreactor system as claimed in claim 16, wherein, for at least one of the at least one solar collector, at least one of the at least one energy supply component, to which the solar collector is operatively coupled, includes a light transmission component configured for transmitting light energy, derived from at least a fraction of the received incident solar radiation, and effecting its supply to the reaction mixture disposed within the reaction zone of the photobioreactor to thereby effect exposure of the reaction mixture to photosynthetically active light radiation.

22. The photobioreactor system as claimed in claim 16, wherein the photobioreactor includes a reaction zone including a minimum vertical extent that is greater than the diameter or the width of the reaction zone.
